(19) United States
(12) Patent Application Publication
(10) Pub. No.: US 2025/0243531 A1
(43) Pub. Date: Jul. 31, 2025

(54) METHODS FOR COMBINING IN SITU SINGLE CELL DNA AND RNA SEQUENCING

(71) Applicant: ARIZONA BOARD OF REGENTS ON BEHALF OF ARIZONA STATE UNIVERSITY, Scottsdale, AZ (US)

(72) Inventors: Kerry GEILER-SAMEROTTE, Scottsdale, AZ (US); Kara SCHMIDLIN, Mesa, AZ (US); Leandra BRETTNER, Mesa, AZ (US)

(21) Appl. No.: 18/719,471

(22) PCT Filed: Dec. 14, 2022
(86) PCT No.: PCT/US2022/081578

§ 371 Date: Jun. 13, 2024

Related U.S. Application Data
(60) Provisional application No. 63/289,269, filed on Dec. 14, 2021.

(51) Int. Cl. C12Q 1/6841; C12N 15/10

(52) U.S. Cl. CPC C12Q 1/6841; C12N 15/1065

(57) ABSTRACT

Disclosed herein is an in situ, high throughput, single-cell region(s) of interest (ROI) or whole-genome sequencing technology developed for sequencing gene(s) or genomes in large heterogeneous cell populations combined with single-cell RNA sequencing. More specifically, the technology disclosed herein does not require cell sorting or isolation because it uses the cell membrane to separate each genome whereupon single genomes and transcriptomes are concurrently sequenced. While other in situ single-cell sequencing technologies are only able to work with RNA, and must first convert DNA to RNA, the method of this disclosure operates directly on DNA as well as on RNA. Thus, it simultaneously sequences each single cell's genome and transcriptome.

### CROSS-REFERENCE TO RELATED APPLICATIONS

The present application claims priority to U.S. Provisional Patent Application No. 63/289,269 that was filed Dec. 14, 2021, the entire contents of which are hereby incorporated by reference.

### STATEMENT REGARDING FEDERALLY SPONSORED RESEARCH OR DEVELOPMENT

This invention was made with government support under R35GM133674 awarded by the National Institutes of Health. The government has certain rights in the invention.

### SEQUENCE LISTING

A Sequence Listing accompanies this application and is submitted as an xml file of the sequence listing named “112624_01376.xml” which is 2,929 bytes in size and was created on Dec. 14, 2022. The sequence listing is electronically submitted via Patent Center and is incorporated herein by reference in its entirety.

### FIELD

The field of the invention relates to methods for single-cell sequencing of genomic DNA and single-cell sequencing of the transcriptome.

### BACKGROUND

With the advent of Next Generation DNA sequencing and the “omics” era, technology for studying the genetic content and function of biological systems has rapidly advanced. Initially, genomics and transcriptomics studies were performed on populations or “batch cultures”. The resulting data represent an average across all cells, ignoring any diversity in a population. However, single-cell techniques permit the study of heterogeneity within populations and are revealing the extent to which variation contributes to biological behaviors. In order to associate sampled genetic sequences with a given cell, the genetic material is often labelled with a DNA barcode sequence that is unique to each cell. In the first generation of single-cell technologies, barcodes were added after individual cells were sorted into separate containers, such as 100 uL wells or microfluidic droplets. This is limiting for several reasons, one of which is that separating cells is difficult and time consuming. Another is that it is difficult to wash or quench each of these exceedingly tiny single-cell reactions in order to perform subsequent reactions. Thus, no methods currently exist to amplify and barcode genomic DNA for single-cell genomic DNA sequencing in combination with single-cell RNA sequencing, (scRNA-seq), as this would require two sequential reactions. Further, no method exists to collect barcoded DNA from single cells along with any barcoded cDNA.

### SUMMARY

In a first aspect of the current disclosure methods are provided. In some embodiments, the methods comprise: a) contacting a plurality of fixed and permeabilized cells comprising genomic DNA and cellular RNA with (i) a first set of DNA amplification primers configured to amplify genomic DNA, and (ii) a DNA polymerase; wherein the DNA amplification primers comprise a design selected from (a) or (b), or a combination of (a) and (b) to generate DNA amplicons: (a) (i) a first universal linker sequence (1-ULS); wherein each primer comprises the same 1-ULS sequence; (ii) optionally, a first well-specific barcode sequence (1-BC); wherein the primers in each well comprise a different 1-BC sequence; (iii) random hexamers which hybridize to complementary sequences on genomic DNA of the cells; (b) (i) the first universal linker sequence (1-ULS); wherein each primer comprises the same 1-ULS sequence; (ii) optionally, the first well-specific barcode sequence (1-BC); wherein the primers in each well comprises a different 1-BC sequence; (iii) a sequence that is designed to hybridize to a specific sequence on the genomic DNA of the cell; and amplifying the DNA; b) quenching the polymerase used for the amplifying of step a); c) contacting the plurality of fixed and permeabilized cells with a mixture comprising (i) reverse transcriptase; (ii) a first set of reverse transcription primers wherein the set comprises (a) the first universal linker sequence (1-ULS); wherein each primer in the first primer set comprises the same 1-ULS sequence; (b) a well-specific barcode sequence (2-BC); wherein the primers in each well comprise a different 2-BC sequence, and optionally wherein, the 2-BC sequence is the same as the 1-BC sequence; (c) a target hybridization region comprising oligo dT sequences or random hexamer sequences; d) reverse transcribing the RNA present in the cell to generate cDNA; e) barcoding the products of step a) and d) with sequential rounds of split and pool to uniquely label the amplified nucleic acid of each cell, wherein the last round of barcoding comprises ligating an oligonucleotide comprising a ULS, a well-specific barcode, an affinity moiety, and common primer sequence to generate barcoded amplicons comprising the affinity moiety and barcoded cDNA comprising the affinity moiety; f) lysing the plurality of cells to release the barcoded amplicons comprising the affinity moiety and the barcoded cDNA comprising the affinity moiety; g) capturing the released amplicons by contacting the barcoded amplicons comprising the affinity moiety and the barcoded cDNA comprising the affinity moiety with an affinity capture reagent. In some embodiments, the methods further comprise: performing a template switch reaction by contacting the captured amplicons and cDNA with (i) reverse transcriptase, and (ii) template switch primers comprising at least three consecutive riboguanosine nucleotides and a terminal primer sequence. In some embodiments, the methods further comprise generating fully double-stranded captured amplicons and cDNA, wherein the fully double-stranded amplicons and cDNA comprise the terminal primer sequence. In some embodiments, generating fully double-stranded captured amplicons comprises contacting the captured barcoded amplicons and cDNA with primers that hybridize to the specific sequence on the amplicons, and a DNA polymerase. In some embodiments, generating fully double-stranded captured amplicons comprises ligating a double-stranded DNA sequence comprising the terminal primer sequence to the free end of the captured amplicons. In some embodiments, generating fully double-stranded captured amplicons comprises contacting the captured barcoded amplicons and cDNA with an enzyme comprising polymerase activity, and oligonucleotides, wherein the oligonucleotides comprise random hexamers and the terminal primer sequence, wherein the oligonucleotides are configured to produce double-stranded barcoded amplicons comprising the terminal primer sequence. In some embodiments, the methods further comprise amplifying the fully double-stranded amplicons and cDNA to generate free amplification products. In some embodiments, the methods further comprise sequencing the free amplification products. In some embodiments, the set of primers are selected from only design (a). In some embodiments, the set of primers are selected from only design (b). In some embodiments, the set of primers are selected from a combination of designs (a) and (b). In some embodiments, the amplification of step a) comprises isothermal amplification. In some embodiments, the temperature of the isothermal amplification reaction is about 20-40° C. In some embodiments, the temperature of the isothermal amplification reaction is about 20-30° C. In some embodiments, the temperature of the isothermal amplification reaction is about 30-40° C., or about 40-50, or about 50-60, or about 60-70, or about 62-68° C. In some embodiments, the temperature of the isothermal amplification reaction is about 30° C. In some embodiments, step a) is incubated for about 30 minutes to about 24 hours. In some embodiments, step a) is incubated for about 16 hours. In some embodiments, step a) comprises contacting the plurality of fixed and permeabilized cells with an isothermal polymerase. In some embodiments, step a) comprises contacting the plurality of fixed and permeabilized cells with phi29 polymerase. In some embodiments, the concentration of phi29 polymerase is about 400 units/ml. In some embodiments, step a) comprises contacting the plurality of fixed and permeabilized cells with a crowding agent. In some embodiments, the crowding agent comprises one or more of: polyethylene glycol 8000 (PEG-8000), trehalose, and sorbitol. In some embodiments, the crowding agent is PEG-8000. In some embodiments, the concentration of PEG-8000 is about 7.5% volume/volume. In some embodiments, the crowding agent is trehalose. In some embodiments, concentration of trehalose is about 0.4 M. In some embodiments, the crowding agent is sorbitol. In some embodiments, the concentration of sorbitol is about 0.5 M. In some embodiments, the oligonucleotides of step e) are ligated to the products of a) and d) with T4 DNA ligase. In some embodiments, the lysis of the plurality of cells of step f) comprises contacting the cells with sodium dodecylsulfate (SDS). In some embodiments, the lysis of the plurality of cells of step f) comprises contacting the cells with proteinase K. In some embodiments, the affinity moiety and capture reagent of step g) comprise biotin and streptavidin. In some embodiments, the affinity moiety and capture reagent of step g) comprise digoxigenin and anti-digoxigenin antibody. In some embodiments, generating fully double-stranded captured amplicons and cDNA comprises contacting the captured barcoded amplicons and cDNA with phi29 polymerase. In some embodiments, the concentration of phi29 polymerase is about 400 units/ml. In some embodiments, generating fully double-stranded captured amplicons comprises isothermal amplification. In some embodiments, the temperature of the isothermal amplification reaction is about 20-40° C. In some embodiments, the temperature of the isothermal amplification reaction is about 20-30° C. In some embodiments, the temperature of the isothermal amplification reaction is about 30-40° C., or about 40-50, or about 50-60, or about 60-70, or about 62-68° C. In some embodiments, the temperature of the isothermal amplification reaction is about 30° C. In some embodiments, the amplification reaction is incubated for about 30-120 minutes. In some embodiments, the methods comprise contacting the fully double-stranded amplicons and cDNA with a crowding agent. In some embodiments, the crowding agent is selected from the group consisting of: polyethylene glycol 8000 (PEG-8000), trehalose, and sorbitol. In some embodiments, the crowding agent is PEG-8000. In some embodiments, the concentration of PEG-8000 is about 7.5% volume/volume. In some embodiments, the crowding agent is trehalose. In some embodiments, the concentration of trehalose is about 0.4 M. In some embodiments, the crowding agent is sorbitol. In some embodiments, the concentration of sorbitol is about 0.5 M. In some embodiments, the methods further comprise amplification of the free double-stranded amplicons and cDNA using polymerase chain reaction (PCR). In some embodiments, the free amplification products are purified. In some embodiments, the free amplification products are purified using solid phase reversible immobilization (SPRI) selection.

### BRIEF DESCRIPTION OF THE DRAWINGS

FIGS. 1A and 1B. A) An overview schematic representation of the novel method of this disclosure, which links scDNAseq with scRNAseq inside of the same single cell. For a more detailed schematic representation, see FIGS. 2 and 3. B) Dot plot showing one problem that can happen when combining scDNAseq with scRNAseq. The problem is that the DNA polymerase (e.g., phi29) required during scDNAseq can interfere with the results of scRNAseq. The way it interferes is by amplifying cDNA such that the transcriptomes of cells that underwent combined scDNAseq+scRNAseq would look different than those that have only undergone scRNAseq. The inventors contend with this issue by quenching the phi29 polymerase.

FIGS. 2A, 2B, and 2C A detailed schematic representation of the first half of the novel method of this disclosure. A) First, the inventors diffuse reagents (e.g., phi29 polymerase and optionally barcoded primers) inside of fixed permeabilized cells where genomic DNA is amplified in situ with primers that target a region of interest (ROI) and/or the entire genome. Each amplicon is tagged with a well-specific barcode. This is done either by using barcoded primers (as shown in the figure) or by annealing a barcode onto a ubiquitous annealing sequence that is added to the amplicons via the primer. B) Cells are heated to quench the polymerase and new reagents are diffused into the cells including a reverse transcriptase and well-specific barcoded primers that target RNA. The set of 96 barcodes on these RNA-specific primers can differ from those used to tag genomic DNA. Thus, molecules that were amplified from the genome can possess different barcodes than those that were reverse transcribed from RNA. Finally, cells from all 96 wells are pooled into one tube. C) Pool of cells is split back into a 96-well plate where each well contains a different barcode. These “round 2” barcodes are diffused inside of the cells and annealed to the amplicons. This is repeated several times. The terminal barcode contains a UMI and biotin bead. The UMI helps distinguish unique in situ amplification events from copies that were made during downstream PCR amplification steps.

FIGS. 3A, 3B, 3C, 3D, 3E, and 3F. A detailed schematic representation of the second half of the novel method of this disclosure. A) The final pool of cells from FIG. 2C is lysed. B) Bead-bound DNA and cDNA is purified, meaning any molecules that are not bead-bound are washed away. C) cDNA is prepared for copying off the bead using a template switch reaction. D) DNA is prepared for copying off bead using one of three methods (described further in the main text). E) All molecules are then copied off of the beads and further amplified. F) Amplicons are fragmented and prepared for sequencing by attaching Illumina adaptors.

FIG. 4. A gel image showing that the isothermal phi29 polymerase cannot amplify DNA after it has been heat inactivated at 65 degrees Celsius for 10 minutes.

### DETAILED DESCRIPTION

Sequencing platforms are now capable of delivering enormous amounts of high-quality data. This allows for the possibility of sequencing the genomes of thousands of individual cells. However, current methods to isolate and tag single-cell genomes for sequencing are expensive, arduous, and often require specialized equipment. Further, no method allows for simultaneous sequencing of a cell's DNA and RNA. Other methods convert each single cell's DNA to RNA, such that it can be sequenced following similar protocols as those that are used to study the transcriptome (see reference 12). These methods are only able to isolate barcoded cDNA from single cells, not DNA. The method of this disclosure is inherently different because it works with DNA without ever converting it to RNA or cDNA. The method of this disclosure also works with cDNA. Barcoding both DNA and cDNA, and then extracting barcoded DNA and cDNA molecules from cells are unique attributes of the method disclosed here. Thus, the inventors have developed a new high-throughput method to amplify and sequence single-cell genomes in conjunction with sequencing their transcriptomes. Since the genome represents the genotype, and the transcriptome represents the phenotype, this method simultaneously maps genotype to phenotype for single cells. This is important because a major goal of biology is to understand how genetic changes (e.g., mutations) manifest in differences between organisms. A common way to quantify differences between cells is to look at their transcriptomes. The method of the instant disclosure does not require cell isolation or specialized equipment beyond typical molecular biology laboratory standards and, thus, is user-friendly and scalable, allowing multiplexing of single cells from many different growth conditions or genetic backgrounds.

Sequencing the genome (or a part of the genome) and transcriptome of single cells opens up a number of novel uses and opportunities.

Example 1 of novel uses and opportunities that become accessible due to the novel method of this disclosure: There are regions of the human genome that are hypervariable, such as VDJ regions in T and B cells, but it is unclear how this variation affects cell function. The inventors' method provides a new way to investigate the relationship between these hypervariable regions and cell biology.

Example 2 of novel uses and opportunities that become accessible due to the novel method of this disclosure: Engineered populations of microbes often possess variation in a specific region that serves to distinguish one strain from another. Sometimes this region is referred to as a “barcode”. If researchers want to understand how the transcriptome differs across different strains, they have to express this barcode so that it can be captured via single-cell RNA sequencing (scRNAseq). This is difficult and can have consequences on the transcriptome. The inventor's method resolves this issue by allowing researchers to study strains that are differentiated by barcodes that are not expressed. The inventors amplify the barcode directly from the genome, and then also amplify the transcriptome. Similarly, in cases where strains are not differentiated by an engineered barcode, their entire genome can be sequenced along with the transcriptome to determine which strain matches which transcriptome.

Example 3 of novel uses and opportunities that become accessible due to the disclosed methods: There is much interest in gaining a more detailed picture of the microbes that inhabit specific environments, including the human microbiome. Current single cell RNA-seq methods miss much of the genetic diversity that separates strains because it can only detect regions of the genome that are expressed. The novel methods of this disclosure allow sequencing of the genomes and transcriptomes of single cells. Alternatively, the methods allow sequencing a region of interest (ROI) for example, a ribosomal protein that is commonly used to distinguish strains, as well as the transcriptome. This gives a clearer picture of the inhabitants of microbial communities.

Example 4 of novel uses and opportunities that become accessible due to the novel method of this disclosure: The novel methods allow investigators to determine the distribution of heterogeneous genomes in a population of cells (e.g., a tumor), as well as the extent to which those genetic differences manifest in phenotypic differences.

Most single-cell sequencing methods require physical isolation of individual cells, which is extremely labor intensive and/or costly. The disclosed novel methods do not require physical isolation, but rather leverage the cell membrane to contain and separate the DNA and RNA from individual cells. In other words, this novel platform uses the cell itself as the container for the reactions. The inventors build upon their prior scDNAseq platform using the cell as a container for its own DNA, disclosed in International Pat. App. No. PCT/US2022/040373, which is incorporated herein by reference in its entirety, by incorporating single-cell RNA sequencing (scRNAseq) into the workflow-disclosing a first of its kind scDNAseq and scRNAseq combined platform.

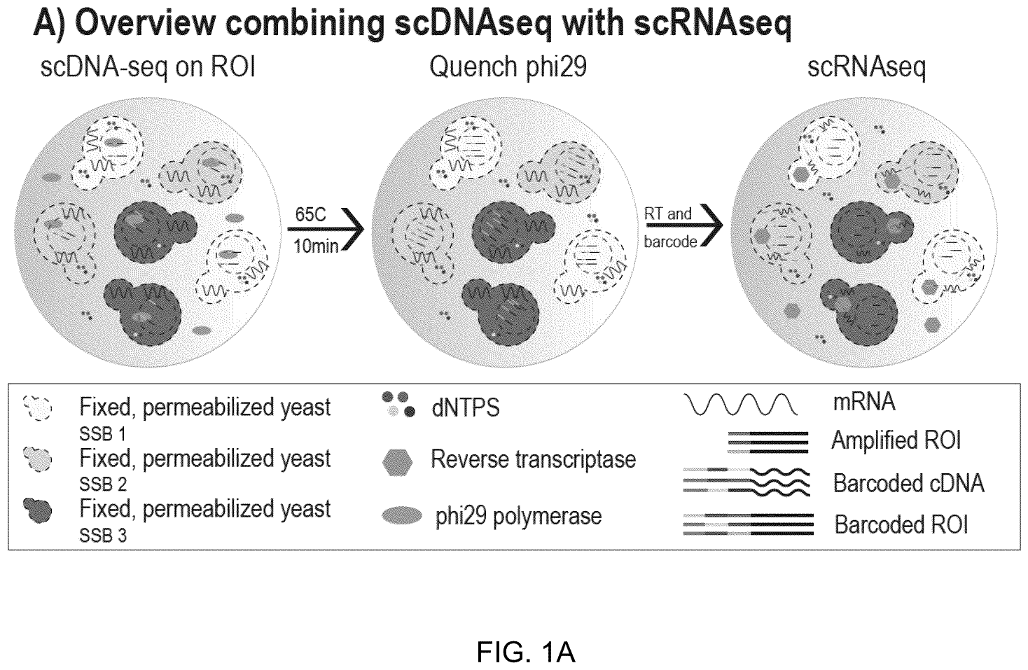

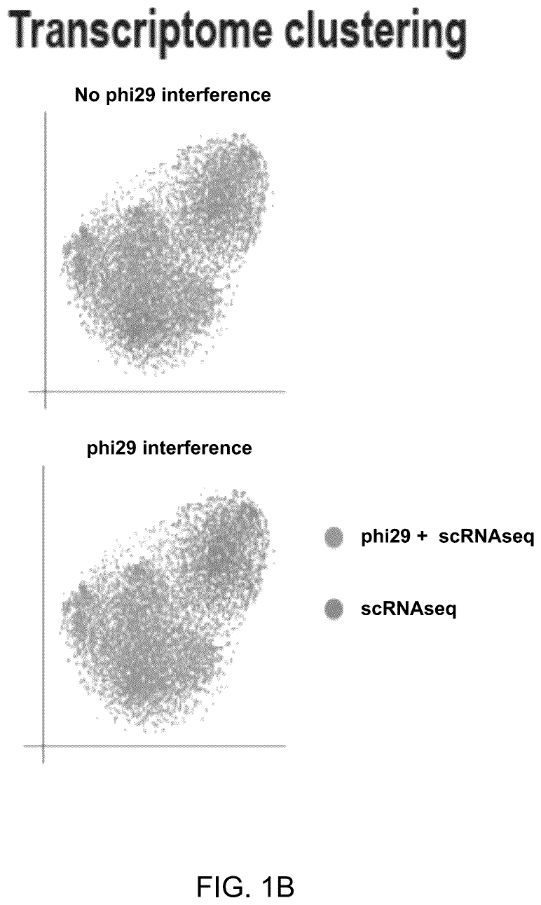

Building upon technologies that allow in situ single-cell DNAseq, disclosed in International Pat. App. No. PCT/US2022/040373, as well as technologies that allow in situ single-cell RNAseq (see references 1, 2, 4, 5, 6, 7, and 8, also see references U.S. Patent Pub. No. US20200263234A1, and U.S. Pat. No. 10,900,065B2 and U.S. patent application Ser. No. 16/949,949), the novel method combines both reactions, including, (1) a genome sequencing reaction and (2) a transcriptome sequencing reaction, inside of a single cell. There are several pitfalls preventing the development of novel combination in situ DNA and scRNA seq methods, which are described in more detail below. Briefly, there are four main problems: 1) the components of the first reaction can interfere with the second reaction (FIG. 1B), the products of the first reaction can be confused with the products of the second reaction, i.e., no current method exists to prepare mixed pools of amplified genomic DNA and cDNA for sequencing, and it would be extremely costly to design barcoded primers for amplifying genomics regions of interest (ROI) following previous approaches. The novel method of this disclosure solves all of these problems as follows:

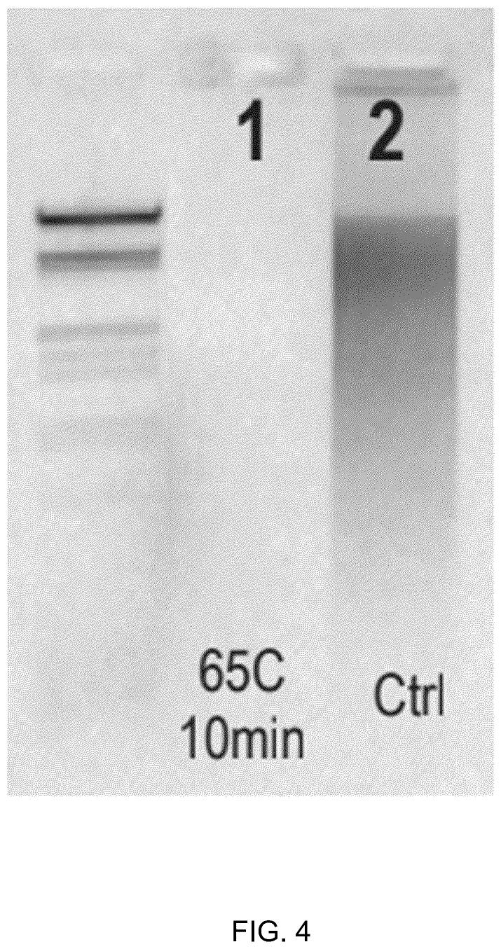

Problem 1: the components of the first reaction can interfere with the second reaction (FIG. 1B). The inventors prior work allowing in situ single-cell DNAseq uses an isothermal polymerase, for example, phi29 polymerase, Klenow exo-DNA Polymerase I, Bsu polymerase, Bst polymerase, Bsm polymerase, to amplify the genome or a part of the genome within the cell (3). In situ genome amplification is a critical first step to produce enough genomic DNA for sequencing. But the presence of this isothermal polymerase creates problems with downstream RNA sequencing. RNA molecules do not need to be amplified in situ for single-cell experiments as those sequences are used to generate gene expression counts. In fact, amplification is a detriment, as it skews transcriptional profiles (FIG. 1B). Thus, the isothermal polymerase must be deactivated so that it does not have the opportunity to amplify the cDNA created during the procedure to prepare the transcriptome for sequencing. Inactivating the polymerase with heat is effective at quenching its ability to amplify genetic material (FIG. 4). After heat inactivating, a reverse transcriptase (RT) must be added to cells to convert the RNA that comprises their transcriptomes into cDNA. Heat inactivation, as well as addition of RT, would be very difficult to do in the context of most single-cell sequencing methods. The problem is that these methods require physical separation of cells. Since every cell is isolated into a separate reaction vessel, this would mean that thousands or hundreds of thousands of reaction vessels would need to be heated, and subsequently have RT added. Adding subsequent chemicals and enzymes to each cell container would be time and labor intensive, may require specialized equipment, and may be impossible in the case of droplet-based cell isolation methods. The novel methods of this disclosure, do not require physical separation of cells, allow easy addition of subsequent reaction components to the cell milieu, and even allow the cells to be washed, removing reaction components that are incompatible. The novel methods of this disclosure also allow easy low heat inactivation (e.g., at about 65° C.) of the isothermal polymerase which allows the cells to remain intact for downstream preparation of RNA for sequencing. The disclosed methods use the cell itself as a container for its genetic material and, thus, all the cells can be combined into one, or a small number of, vessels, e.g., wells, tubes, etc., prior to heating. Once the isothermal polymerase is deactivated, a reverse transcriptase (RT) can be added to convert the cells RNA to cDNA without concern that the polymerase will amplify the cDNA.

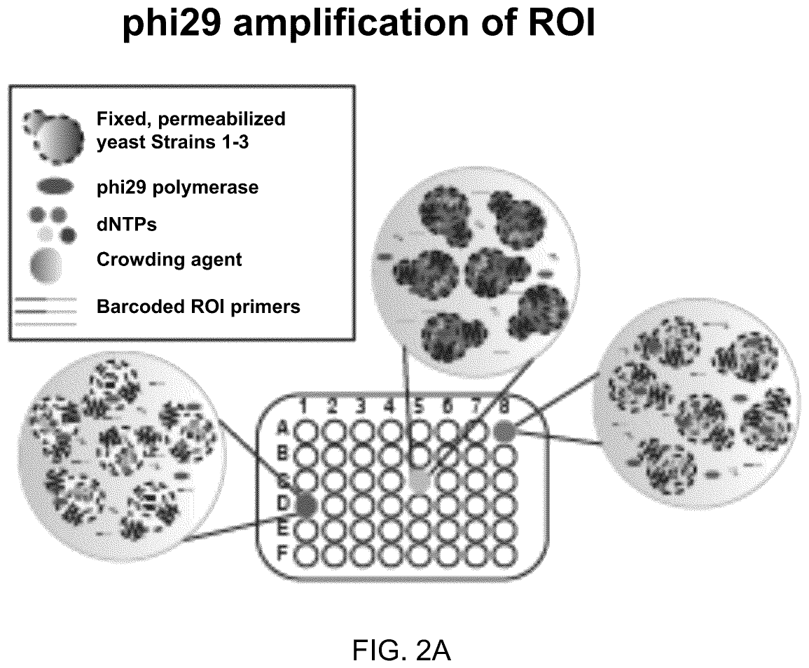

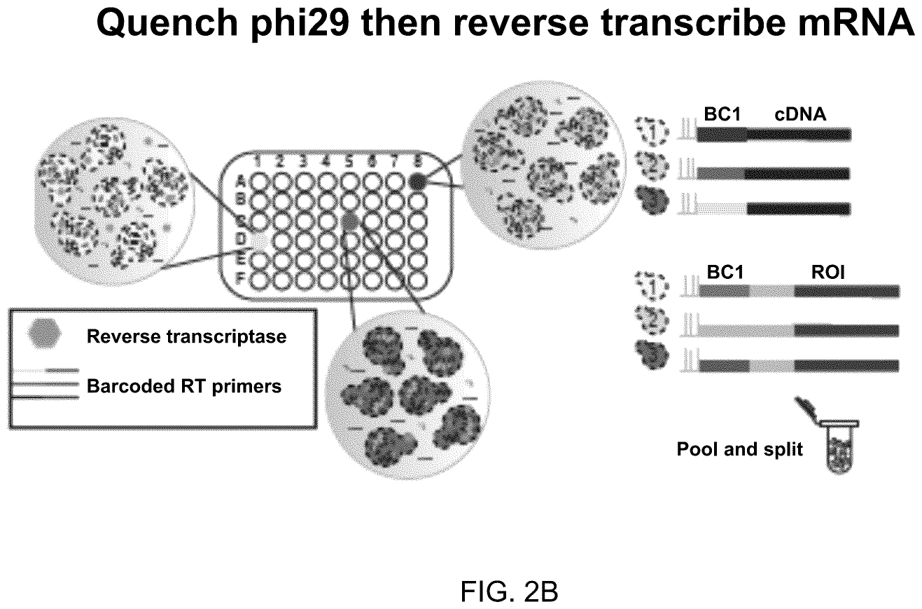

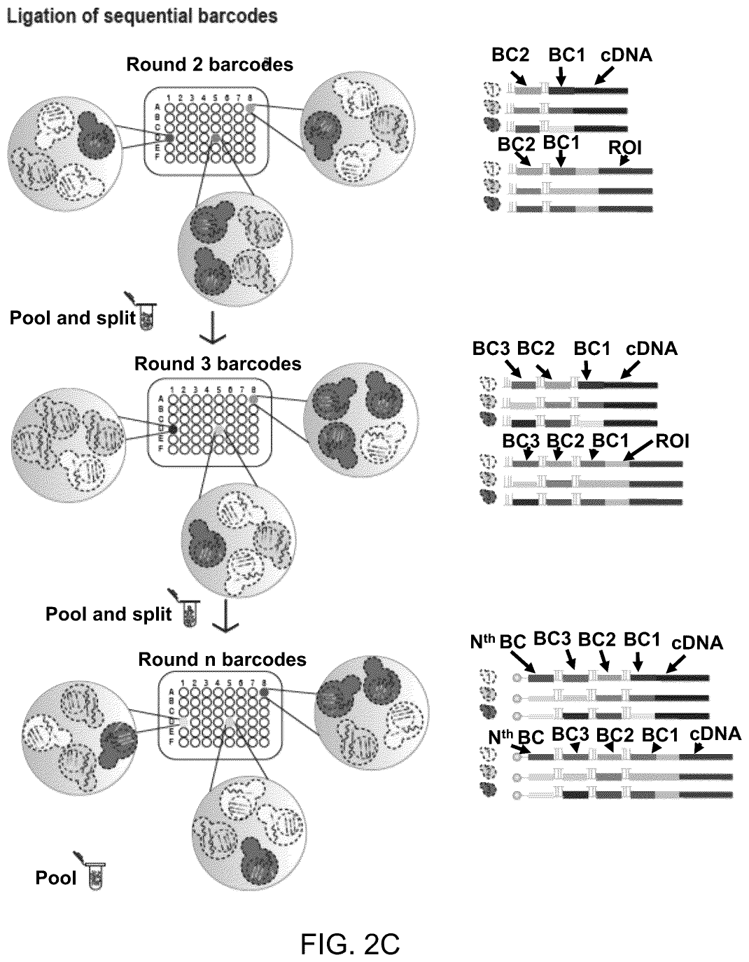

Problem 2: the products of the first reaction (DNA amplicons) can be confused with the products of the second reaction (cDNA): Barcoded primers are used to amplify genomic DNA via the isothermal polymerase, as well as to generate the cDNA sequences created by the reverse transcriptase. After the addition of these first barcodes, the inventors then use an additive combinatorial strategy that sequentially and randomly appends multiple DNA barcodes from an existing pool, creating a unique combination of barcodes that label each cell's genetic material in situ (see FIG. 2C). Thus, the molecules pertaining to each individual cell have unique identifiers. One problem that uniquely happens in cells that are undergoing simultaneous DNAseq and RNAseq, is that the DNA (amplified off the genome) and the cDNA (representing the transcriptome) will have the same barcodes. This creates a problem of determining which molecules represent the genome and which molecules represent expressed genes. To avoid this issue, the novel methods of the disclosure can utilize different barcodes for the first round of DNAseq and the first round of RNAseq (FIG. 2). The first barcode on each molecule thus specifies whether that molecule is DNA or cDNA as well as the well of origin.

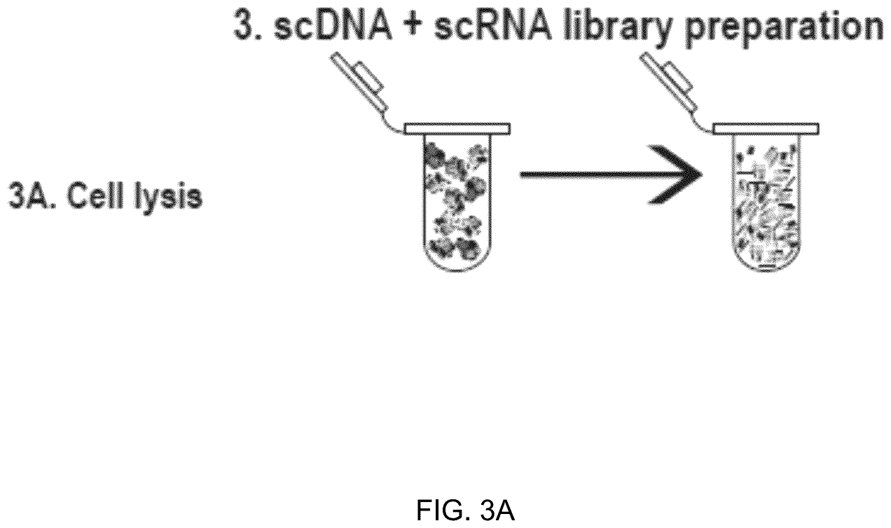

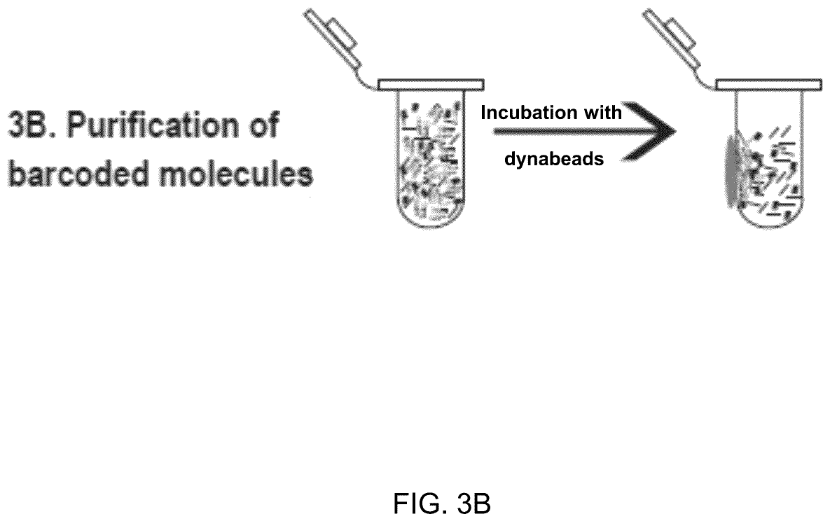

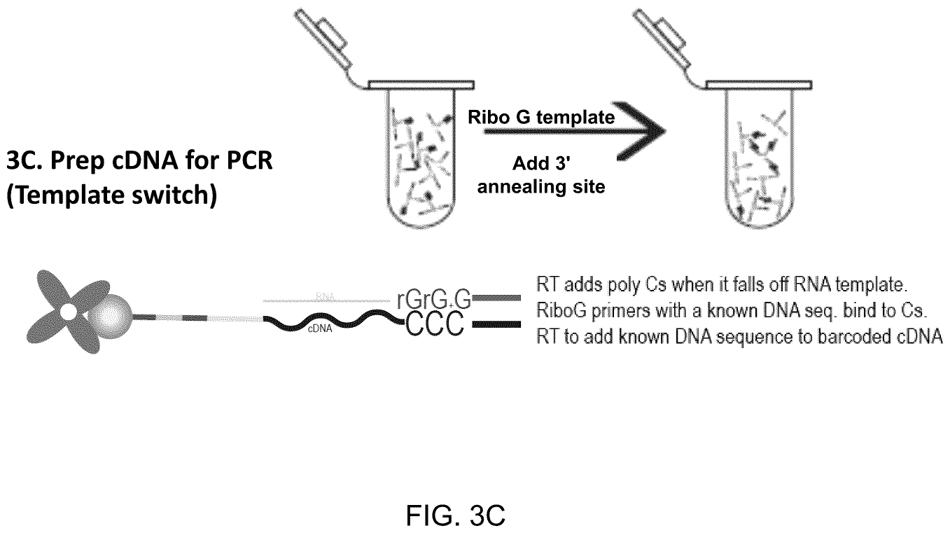

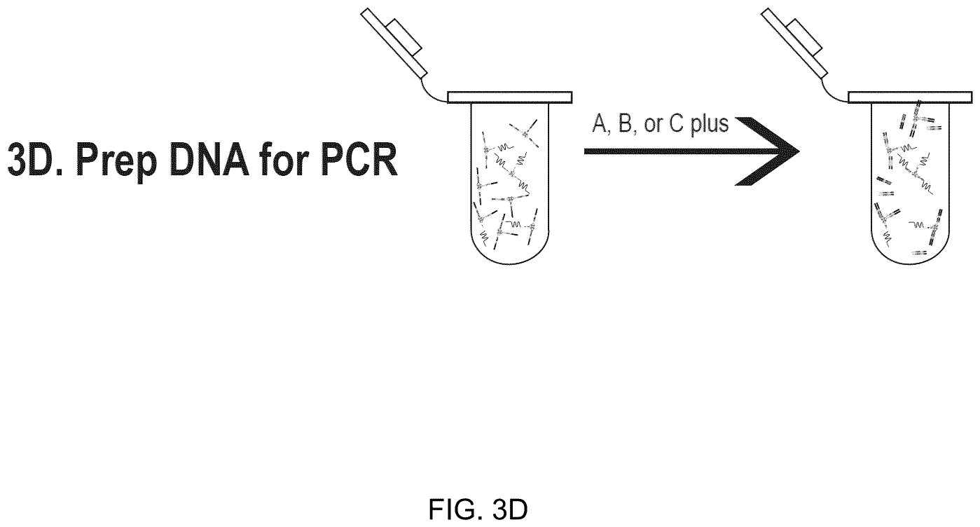

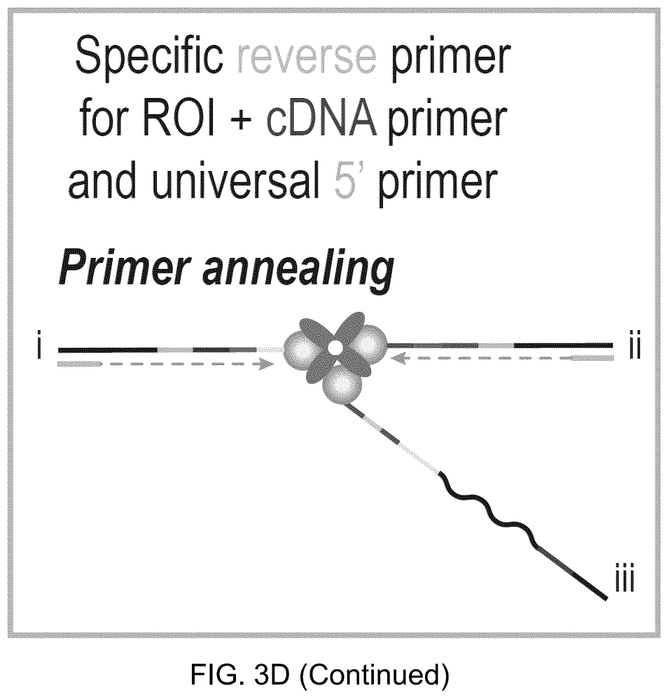

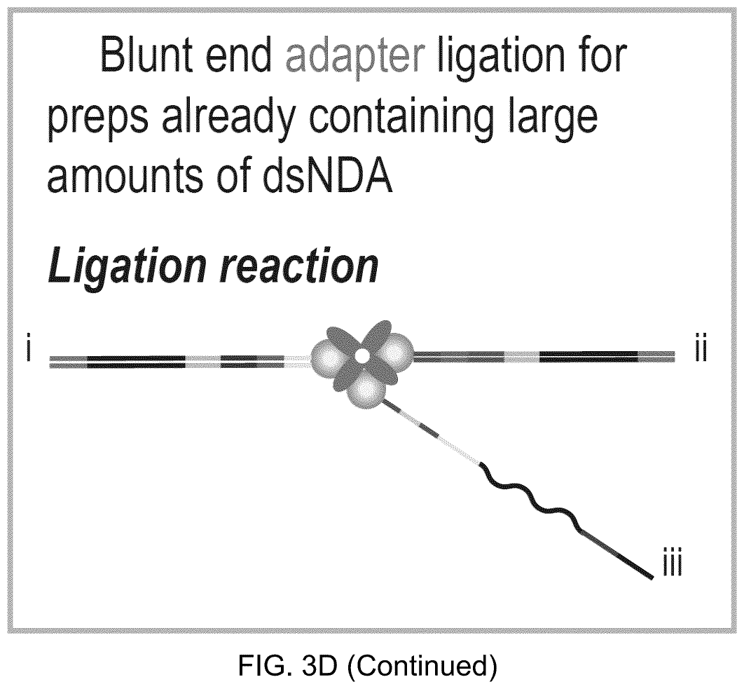

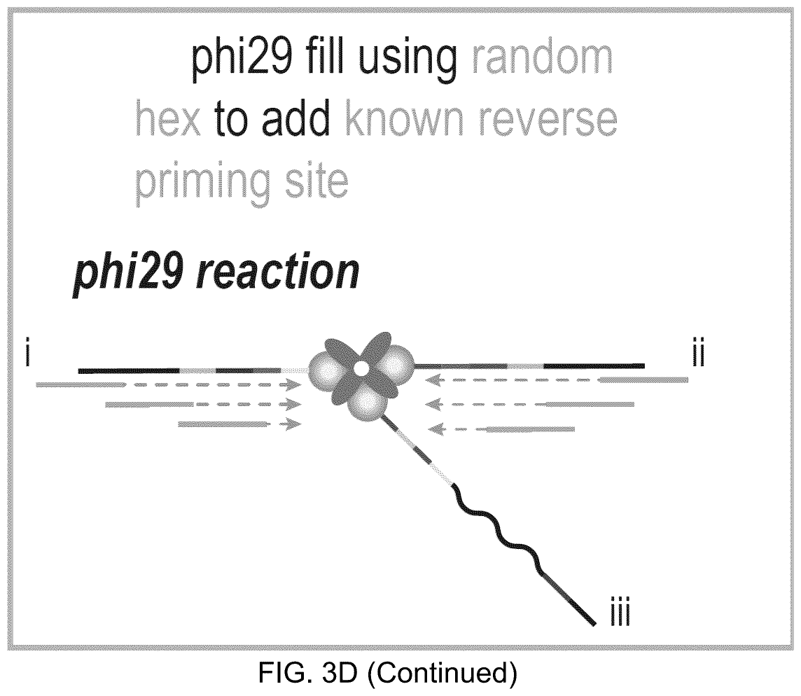

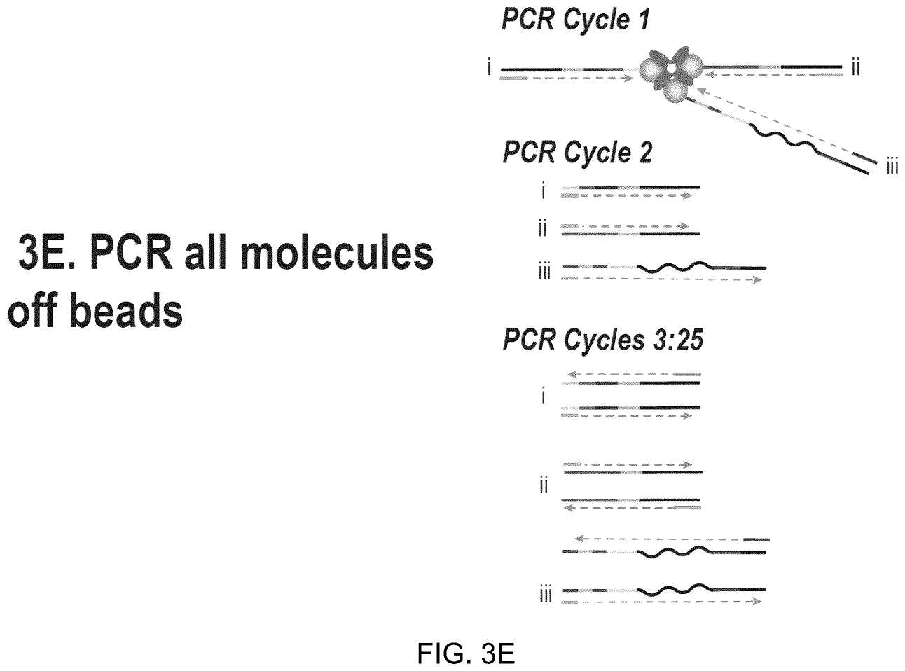

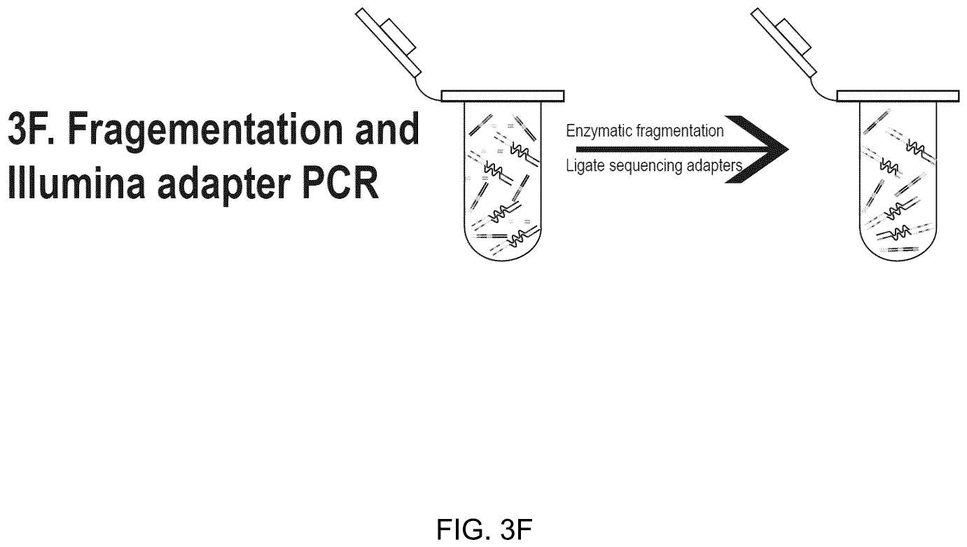

Problem 3: No current method exists to prepare mixed pools of amplified genomic DNA and cDNA for sequencing. After DNA is amplified and given a DNA-specific barcode using an isothermal polymerase (FIG. 2A), and the RNA is reverse transcribed to cDNA and given a cDNA-specific barcode using a reverse transcriptase (FIG. 2B), and additional cell-specific barcodes are appended onto all molecules (including DNA and cDNA) (FIG. 2C), each cell contains a mixed pool of barcoded DNA and cDNA. Because of the different molecular chemistries and experimental needs for processing DNA versus cDNA, the procedure to prepare these mixed pools for sequencing is more complicated than previous methods for in situ single-cell DNA sequencing disclosed in U.S. Provisional Pat. No. 63/233,177, and previous methods for in situ single-cell RNA sequencing. For example, the novel method has separate steps during post-processing to prepare the cDNA for sequencing using a template switch reaction (see FIG. 3C) and then subsequently add a ubiquitous primer adapter to variable sequences at the 3′ end of the barcoded DNA (FIG. 3D). Adding the ubiquitous primer adaptor to variable sequences at the 3′ end of the barcoded DNA can be done in one of three ways (FIG. 3D). The first applies to cases where a small number of genomic regions of interest (ROI) are being amplified from the genome. In these cases, specific primers can be designed that target these regions. The other two methods apply to cases where multiple ROI are being sequenced from the genome or when the entire genome is being amplified. In these cases, if the bead-bound DNA is largely double stranded, the ubiquitous primer adaptor can be ligated on using a blunt end ligase. If the bead-bound DNA is not largely double stranded, then it needs to first be made double stranded by performing a linear PCR reaction seeded, i.e., primed, using random hexamers. These three approaches are described in more detail below but mentioned here to highlight how the novel invention of this disclosure is unique from previous methods.

Problem 4: It would be extremely costly to design barcoded primers for amplifying genomics regions of interest (ROI) following previous approaches. The reason for this is that it would require a very large number of barcoded primers, 96 per each 150 bp ROI. To contend with this issue, the primers used to amplify DNA (FIG. 2A) do not necessarily need to possess barcodes. Instead, they can possess universal linker sequences (ULS). Then the barcodes could be subsequently ligated on. This must be done sequentially, such that first the ROI is amplified with phi29, then DNA-specific first barcodes are ligated onto each amplicon with a barcode specific to each well (FIG. 2A), then the phi29 is quenched, and the rest of the procedure is followed as described in FIGS. 2 and 3.

The following is a detailed explanation of some embodiments of the disclosed methods. The following steps are illustrative in nature and not intended to limit the scope of the disclosure.

To preserve the cellular components, the samples are formaldehyde fixed overnight. Cells are then permeabilized so that membranes can allow enzymes, barcodes and other reagents to pass into the cell to access the genomic DNA (gDNA) as well as RNA.

In some embodiments, the methods for combined scDNA and scRNA sequencing of the current disclosure begin with cells, e.g., prokaryotic or eukaryotic cells, that have been fixed and permeabilized such that reagents may enter and leave through the cell membrane. Critically, cell membrane integrity is preserved, and the nucleic acids present in the cells are sufficiently fixed such that they remain inside the cell and are not dislodged during the procedure.

After the cells are fixed and permeabilized, one then amplifies genomic DNA (or part of the genomic DNA such as a specific gene) in situ via an isothermal polymerase (FIGS. 1A and 2A). In some embodiments the isothermal polymerase is one or more of phi29 polymerase, Klenow exo-DNA Polymerase I, Bsu polymerase, Bst polymerase, Bsm polymerase. In some embodiments, the isothermal polymerase can effectively strand displace and copy DNA at low temperatures (e.g., a temperature lower than required for strand denaturation).

In some embodiments, these reactions are performed in a multi-well plate with each well containing random hexamer primers that bind many places in the genome. The primers contain a well-specific barcode and a universal linker sequence (ULS) at the 5′ end for further barcoding post-amplification. This first barcode, which is incorporated as part of the random hexamer primers, serves as a conditional signifier, because all cells that originate from that initial well are intentionally loaded there. Thus, dozens of separate samples, e.g., cells from different experimental conditions or different subjects, can be processed together. This first barcode also distinguishes amplified DNA molecules from cDNA molecules that are created at a later step of the protocol (FIG. 2A).

In some embodiments, the amplification copies a region of interest (ROI), for example, a specific gene, rather than the entire genome. In some embodiments, such as the field of cancer evolution, an ROI may represent an oncogene, in cases where variation within oncogenes is used to distinguish mutations that give rise to tumor pathogenicity or drug resistance. In some embodiments, the ROI is a strain-specific barcode (SSB). As used herein, “strain-specific barcodes” are strain-specific nucleotide sequences that are integrated into the genome of the organism and are used to distinguish strains in mutant libraries. In such cases where the ROI is an SSB, the amplicon created by amplification of the ROI is optionally given a different first barcode than the cDNA. This is optional because SSB are typically not expressed and so there is no need to distinguish ROI amplified from the genome from ROI found in the transcriptome, as the latter molecules do not exist. In other embodiments, the ROI is a gene that is expressed in the organism and the amplicon produced by amplifying the ROI is given a different first barcode to facilitate differentiation between genomic ROIs and expressed cDNAs derived from mRNA. In embodiments where an ROI is being amplified, the amplification primers may or may not contain a well-specific barcode. The reason for this is that it is expensive to design barcoded primers that targets many ROIs. The primers still do contain a universal linker sequence (ULS) at the 5′ end for further barcoding post-amplification. In cases where the primers do not possess a barcode, one is immediately ligated post-amplification using the ULS. In addition to a well-specific barcode, this ligated sequence also contains a ULS at the 5′ end for further barcoding.

In some embodiments, the primer used to initiate amplification of the ROI comprises a barcode. In other embodiments, the primer used to initiate amplification of the ROI does not comprise a barcode and only has an ULS to allow subsequent annealing of barcodes.

In embodiments where the entire genome is amplified, and in embodiments where one or more ROI is amplified, the first barcode, whether incorporated as part of the primer used to amplify DNA, or ligated on post amplification, serves as a conditional signifier that tells in which well on the 96-well plate a cell originated. Thus, 96 samples can be multiplexed in the same experiment.

In summary of the first step (FIG. 1A), to amplify and barcode the genomic DNA, the fixed and permeabilized cells may be contacted with primers that hybridize with a target region of the genome. In some embodiments, the target region is a region comprising a strain-specific barcode (SSB). In some embodiments, the target region may comprise a region of DNA that is transcribed by the cell. The primer may comprise a region that is complementary to the region of interest and may also comprise a barcode sequence. The cell is further contacted with phi29 polymerase which catalyzes the extension of the target region, resulting in the generation of an amplicon comprising the target region of genomic DNA and the barcode sequence.

After amplifying either the genome (with random hexamer primers) or one or more specific portions of the genome (ROI/ROIs), the DNA polymerase must be quenched (FIG. 1A). As used herein, “quenched” refers to the process of inactivating the polymerase. In some embodiments, quenching comprises inactivation by heat. In some embodiments, quenching comprises incubating the sample containing the polymerase at 65° C. for 10 minutes. Quenching of the phi29 polymerase prevents the enzyme from catalyzing any unwanted extension or amplification in the subsequent scRNA sequencing steps. Next, the cells may be, in some embodiments, washed to remove the DNA target-specific primers and excess phi29 polymerase. The inventors demonstrate successful quenching of the polymerase in FIG. 4.

Following generation of the genomic DNA amplicons and the quenching and washing steps, the mRNA present and fixed in the cells may be reverse transcribed. As used herein, “reverse transcription” refers to generation of complementary DNA (cDNA) from an RNA template. Reverse transcription is performed by an RNA-dependent DNA polymerase enzyme called “reverse transcriptase”. Several reverse transcriptases are known in the art and are suitable for the disclosed methods. In some embodiments, the reverse transcriptase used in the disclosed methods is, for example, murine Moloney leukemia virus (MMLV) reverse transcriptase. Similarly to DNA-dependent DNA polymerases, RNA-dependent DNA polymerases or reverse transcriptases require, in some embodiments, divalent cations, dNTPs, and additional reaction components, and the like, in order to properly function. Methods of preforming reverse transcription are well known in the art. In some embodiments, the reverse transcription reaction may be performed by contacting the cells with reverse transcriptase enzyme and oligo dT primers comprising a well-specific barcode and a universal linker strand sequence. Then, the reverse transcription reaction may be allowed to proceed resulting in generation of cDNA that comprises the well-specific barcode and universal annealing sequence (ULS) (FIGS. 1A and 2B). To distinguish the cDNA from DNA, the barcodes added at this step can be different than those in the previous step where amplicons generated from genomic DNA are barcoded. But all barcodes, including those added in this and the previous step, share the same annealing sequence (ULS) that allows additional barcodes to be appended.

At this point in the exemplary procedure, every cell contains amplified and barcoded genomic DNA as well as barcoded cDNA. The next challenge is adding additional barcodes to both the DNA and the cDNA such that every single molecule ends up with a combination of barcodes that can be used to trace sequenced molecules back to their cell-of-origin. For this, split and pool methods may be used to generate uniquely barcoded genomic DNA (gDNA) amplicons and uniquely barcoded cDNA molecules within every cell (FIG. 2C). During split and pool, the barcodes that are appended to the cDNA molecules are the same as those appended to the DNA molecules. Therefore, both the gDNA amplicons and cDNA can be traced back to the cell from which they originated using informatics. Briefly, after the first barcode is added to DNA amplicons (FIG. 2A) and cDNA (FIG. 2B), the cells may be “pooled” into a single vessel and re-distributed into new wells, i.e., “split”. In some embodiments, the “pooling” step may involve removing cells from each occupied well of a 96-well plate and “pooling” the cells in a single tube and mixing. In some embodiments, the “splitting” step may involve “splitting” the “pooled” cells into a new multi-well container where each well contains 1) DNA ligase, and 2) a short, well-specific barcode sequence with a complimentary adapter to ULS and an additional 5′ ULS to allow further barcoding. A ligation reaction covalently bonds these barcodes to the 5′ end of each cell's amplified DNA and cDNA, which adds a second barcode to each molecule (FIG. 2C; top).

In some embodiments, the cells are subsequently pooled and split again into a new multi-well plate where the process is repeated, adding a third barcode (FIG. 2C; middle). Cells that received the same first barcode are unlikely to receive the same second or third barcode. This process is completed an arbitrary number of times depending on the size of the population of cells being processed, as unique barcode combination possibilities scale exponentially with each additional round (e.g. 96 barcodes in a 96 well plate, n split-pools=96″ possible barcode combinations). Each cell is, thus, uniquely labelled by probabilistically biasing the outcome such that it takes its own path through the barcode plates. The final round of split and pool may comprise contacting the cells with 1) DNA ligase, 2) a primer comprising a well specific barcode, common primer sequence, and a ULS that is complementary to the previous ULS, and 3) an affinity moiety. In some embodiments, the affinity moiety may be biotin (FIG. 2C; bottom). As used herein, “common primer sequence” refers to a nucleic acid sequence that is known and can be used as a site to hybridize a primer for amplification of the captured nucleic acids.

At this point in the exemplary method, each cell contains amplified and barcoded copies of its genomic DNA and cDNA and each cell has a unique barcode combination that can be used to identify which molecules, i.e., DNA and cDNA sequences, are from which cell. The cells are then lysed to extract the barcoded DNA and cDNA by incubating with streptavidin coated magnetic beads to retrieve properly barcoded sequences (FIGS. 3A and 3B). All other material is washed away. The resulting molecules present challenges, namely that they are now affixed to the substrate comprising the capture reagent, e.g., a bead. Currently available sequencing library preparation methods are unable to solve these problems. While the beads serve to isolate the molecules one wishes to keep, in order to sequence this DNA and cDNA, one must copy the molecules off of the beads as they are firmly attached via the biotin-streptavidin bond. The way this is done for RNA-seq starts with a template switch reaction, but this method is incompatible for use with genomic DNA because the chemistry is different. As used herein, “template switch” or “template switch reaction” refers to the use of the intrinsic property of some reverse transcriptases which add non-templated ribocytosines to the cDNA molecule. Therefore, a primer comprising riboguanosines and additional sequences (e.g., terminal primer sequence) can be annealed to the cDNA and added via the reverse transcriptase. In the novel method, the inventors first perform a template switch reaction to prepare the cDNA to be copied off the beads (FIG. 3C). After this step, the cDNA comprises the terminal primer sequence. As used herein, “terminal primer sequence” refers to a sequence that is known and can be used to anneal a primer for amplification. Thus, addition of the terminal primer sequence to cDNA or amplicons allows amplification of the cDNA or amplicons by addition of a primer complementary to the terminal primer sequence. Then, the inventors prepare to copy the DNA off the beads (FIG. 3D). In cases where the DNA represents known regions of the genome, this is done by annealing primers that target that region, similarly to the terminal primer sequence (FIG. 3D; left). Thus, in such cases, addition of the terminal primer sequence may not be necessary. In cases where the DNA represents the entire genome, DNA amplicons are copied off the substrate, e.g., beads, by attaching an intermediate 3′ primer adapter via blunt-end ligation to the unbarcoded end (FIG. 3D; middle). In some embodiments, it is also done by using random hexamer primers with an attached primer adapter region and performing a phi29 reaction on the beads (FIG. 3D; right). Since phi29 is strand displacing, the longest complementary strand will be annealed to the bead-attached DNA. Washing the beads will remove excess or short copies. To amplify all molecules, cDNA and DNA off the beads, traditional exponential PCR can then be performed with primers that target 1) the adapter region in the cell-specific barcode and 2) the regions added or described in FIGS. 3C and 3D (FIG. 3E). At this point, the libraries can be prepared using standard practices because these copies, unlike the originals, are not bound to a streptavidin bead (FIG. 3F).

One potential application of this technology is to help understand diversity of microbial communities like the microbiome. Current methods to understand transcriptional diversity of these communities are limited because only a portion of the genome is transcribed. The portion that is transcribed might not be the portion that can be used to differentiate similar strains. The novel invention allows targeted DNA-seq of an ROI that differentiates strains, combined with transcriptional profiling.

Another potential application of this technology is understanding transcriptional diversity in engineered strains. These strains are often barcoded with a strain specific barcode (SSB) that is not expressed [see, reference 10], or is designed to be expressible which can alter the rest of the transcriptome and runs the risk of not being captured if the level of expression is too low. The novel method allows transcriptional profiling of mutant libraries in high throughput by allowing the SSB to be amplified and sequenced along with the transcriptome so researchers can easily tell which transcriptome belongs to which strain with greater probability of capture and without interfering with the natural expression patterns of the cell.

Another potential application of this technology is in studying cancer transcriptomes. Even in cases when oncogenes that differentiate different tumor types are expressed, they might not be expressed at high enough levels inside of every cell to confidently associated a cell's transcriptome with its genotype. By allowing targeted amplification of an ROI like an oncogene, the novel invention allows transcriptional profiling of tumor diversity while allowing the association of a transcriptional profile with the genotype that has that profile.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

The term “subject” may be used interchangeably with the terms “individual” and “patient” and includes human and non-human subjects. In some embodiments, subjects may be plants, fish, birds, reptiles, or mammals. In some embodiments, the disclosed methods are performed on fungal, bacterial, archaeal, algal, or protozoal cells.

As used herein, “fixation” or “fixing” refers to the process of chemically stabilizing organic, inorganic, or a combination of organic and inorganic molecules through the use of reagents, known as “fixatives”. Exemplary fixatives for the present disclosure include, but are not limited to, formaldehyde, formaldehyde derived from paraformaldehyde, formalin, phosphate buffered formalin, formal calcium, formal saline, zinc formalin, alcoholic formalin, glutaraldehyde, other organic aldehydes, methanol, ethanol, isopropanol, or other organic alcohols, or solutions containing organic alcohols or aldehydes.

As used herein, “permeabilization” or “permeabilizing” refer to the process of introducing openings into barriers to allow the penetration of desired molecules past the aforementioned barrier. In some embodiments, the barrier comprises a cell membrane, and or a cell wall. In some embodiments, permeabilization is performed by, for example, enzymes on biological membranes. Exemplary enzymes for permeabilization of biological membranes include, but are not limited to: proteinase K, lysozyme, and zymolyase. In some embodiments, permeabilization is performed by, for example, detergents on biological membranes.

The term “hybridization” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

As used herein, “amplification” refers to the process of semi-conservatively replicating nucleic acid strands by enzyme-catalyzed extension. Exemplary enzymes for amplification of nucleic acids in the current disclosure include, for example, nucleic acid polymerases. In some embodiments, an isothermal polymerase is used to amplify nucleic acids. In some embodiments, amplification is carried out with a high-fidelity polymerase, such as Q5, with the technique known as polymerase chain reaction (PCR). Amplification can be performed with natural and non-natural nucleotide bases, ribonucleotide bases, or deoxyribonucleotide bases, labeled nucleotide bases, and the like.

As used herein, “isothermal amplification” describes amplification of DNA targets without heat denaturation of DNA. In contrast, polymerase chain reaction (PCR) requires cycling through different temperatures for denaturation, hybridization, and extension, or, in some cases, different temperatures for (1) denaturation and (2) hybridization/extension. Isothermal amplification may be preceded by a higher temperature hybridization step that does not denature the DNA target. Exemplary polymerases useful for isothermal amplification are referred to herein as isothermal polymerases, and include, but are not limited to phi29 polymerase, Klenow exo-DNA Polymerase I, Bsu polymerase, Bst polymerase, Bsm polymerase. Isothermal amplification may take place at, for example, about 20-40° C., about 20-30° C., about 30-40° C., or about 30° C., or about 40-50, or about 50-60, or about 60-70 deg. C. Isothermal amplification may also take place at about 62-68° C., for example, about 62, about 63, about 64, about 65, about 66, about 67, or about 68° C. Isothermal amplification may require incubation times of about 30 minutes to about 24 hours, or about 12 hours to about 24 hours to complete the reaction.

As used herein, “ligation” refers to the joining of two nucleic acid molecules through the formation of covalent phosphodiester bonds, i.e., by forming phosphodiester bonds between a 3′ OH and a 5′ phosphate molecule on the two nucleic acid molecules. Ligation may involve the joining of double-stranded or single-stranded nicked nucleic acid molecules. In some embodiments, two blunt-ended nucleic acid duplexes are ligated together. In some embodiments, two nucleic acid duplexes that have single-stranded regions that are substantially complementary to one another allowing hybridization of the two nucleic acid duplexes are ligated to one another. Suitable ligase enzymes are known in the art including, but not limited to, T4 DNA ligase and T7 DNA ligase.

As used herein, “pooling” refers to the process of taking previously separate samples, such as cells, and combining them to create a “pool” of samples (such as cells) that optionally may be separated bioinformatically and identity determined post-experiment during data analysis.

As used herein, the noun “well” refers to a single container or reaction vessel. Though the term well is often used when referring to plates or microplates, it is to be understood that the methods of the current disclosure may also be performed using, for example, tubes or other vessels capable of containing and separating liquids.

As used herein, “affinity moiety” refers to a chemical constituent, often attached to a molecule of interest that can be specifically recognized and bound by a “capture reagent” with high affinity, and with binding strength suitable to allow purification of the molecule of interest to which the affinity moiety is attached. The use of affinity moieties with capture reagents is collectively referred to as “affinity capture” in the context of separation of molecules of interest using the pair of reagents (affinity capture reagents). In some embodiments, exemplary affinity capture reagents include, without limitation, for example, biotin and streptavidin, digoxigenin and anti-digoxigenin antibodies, antibody-antigen pairs, and covalent click chemistry.

As used herein, “sequencing” refers to the sequencing of nucleic acids. Sequencing of nucleic acids may be accomplished using, by way of example but not by way of limitation, Sanger sequencing, or next-generation sequencing.

As used herein, “barcode” refers to a nucleotide sequence of any length that is used to identify, for example, nucleotide sequences that are derived from a single sample. An exemplary property of a barcode is the ability to distinguish the sequence of the barcode from any known sequence present in the sample, thereby rendering the barcode sequence informatically distinct and permitting identification or quantification of any nucleotide sequence comprising the barcode. In some embodiments, a barcode may be 6-8 nucleotides in length. Each barcode must be detected in a single sequencing “read.” Therefore, barcode length is, in principle, dictated by the sequencing platform used to analyze the samples.

As used herein, “universal linker strand” or “ULS” refers to a nucleotide sequence that facilitates the hybridization of single stranded primers, such that the hybridization partner of the ULS is the reverse complement of the ULS, or substantially similar to the reverse complement of the ULS. In some embodiments, the ULS is 10-20 (inclusive) nucleotides in length. In some embodiments, the ULS is 15 nucleotides in length.

As used herein, “split and pool” refers to a process for introducing complexity into a group of compounds, e.g., nucleic acids, such that the knowledge of the initial source of each compound is preserved and can be determined after the completion of the split and pool process. Split and pool relies on probability to ensure that each individual compound has a high statistical likelihood to take a unique path through a set of steps, with each step introducing a new “barcode” which is linked to the compound. A first “barcoding event”, meaning the attachment, such as by ligation, of a barcode to the compound, is performed with a knowledge of the identity of the compound and the identity of the barcode to which each compound is attached. After each barcoding event, all of the individual compounds are combined, or “pooled”, and “split”, or redistributed into new reaction vessels, with each vessel containing a unique barcode. Thus, a second round of barcoding reduces the chances that two compounds will be split into the same reaction vessel and be attached (e.g., ligated) to the same barcode. Therefore, after successive rounds of splitting and pooling the compounds, each of the compounds is likely to be attached (e.g., ligated) to a unique set of barcodes that correspond to the compound's unique trajectory through the split and pool process. The possible number of unique compounds that can be effectively barcoded using split and pool increases with both the number of reaction vessels, and therefore the number of barcodes, and with the number of successive rounds of barcoding events. Non-limiting examples of potential uses for the split and pool process include the preparation of nucleic acid libraries. As disclosed herein, in one embodiment of the present technology, split and pool may be used to efficiently label nucleic acids that are derived from a single cell with a unique barcode allowing for multiplexed sequencing of nucleic acids derived from many cells.

As used herein, the term “random hexamer” or “random hexonucleotide” refers to a region of six nucleotides in length comprising sequences that are synthesized at random. The purpose of random hexamers is, in most applications, to bind complimentarily to nucleotide sequences of unknown identity. Thus, because random hexamers theoretically cover all possible sequence permutations for a hexameric (6-member) nucleotide, they are likely to bind at many positions to nucleotides of any sequence. It should be understood, however, that a key feature of random hexamers is not that they are six nucleotides in length, but rather that they have random sequence identity. In other words, for many applications it is possible to provide random pentamers (5-member), heptamers (7-member), or other random sequences in place of hexamers. In some embodiments, a random hexamer comprises a part of, or a portion of a larger oligonucleotide, such as an oligonucleotide primer.

As used herein, “primer” refers to a single-stranded oligonucleotide. In some embodiments, a primer is used to initiate semi-conservative replication of nucleic acids. In some embodiments, primers are used to “barcode” nucleic acid sequences of interest. In some embodiments, primers comprise a universal linker sequence (ULS) but do not comprise a barcode. In some embodiments, a barcode is added to sequences comprising primers that comprise a ULS by direct ligation using the ULS as a region of homology for the barcode to anneal to the sequence. By way of example, an oligonucleotide primer may comprise from 5′ to 3′: a universal linker strand, a barcode and a random hexamer sequence. By way of example, an oligonucleotide primer may comprise from 5′ to 3′: a universal linker strand, a random hexamer sequence, and a barcode. In one embodiment of the novel method of the disclosure, the primers that are used to randomly barcode genomic DNA are random hexamer primers. In some embodiments, the barcodes are 8 bp long and the UCLs are 15 bps (e.g., UCL1-BC1-random hexamer, ATCCACGTGCTTGAG-ACTCGTAA-NNNNNNATAAGC (SEQ ID NO: 1)). The specific primer used to amplify a specific region of interest is, in some embodiments, 21 bp long (e.g., UCL1-BC1-TTAATATGGACTAAAGGAGGC (SEQ ID NO: 2)). Primers of other lengths may be acceptable.

As used herein, “crowding agent” refers to compounds that decrease the solvent available to macromolecules, thereby increasing the relative concentration of said macromolecules and altering their properties. In some applications, crowding agents have the effect of increasing enzyme activity and accelerating reactions resulting in faster and potentially more specific assays. In some embodiments, crowding agents may include one or more of polyethylene glycol (PEG), polyethylene glycol 8000 (PEG-8000), trehalose, and sorbitol. In some embodiments crowding agents may include ficoll or dextrans.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids, proteins, and/or other compositions described herein may be purified. As used herein, “purified” means separate from the majority of other compounds or entities and encompasses partially purified or substantially purified. Purity may be denoted by a weight by weight measure and may be determined using a variety of analytical techniques such as but not limited to mass spectrometry, HPLC, spectrophotometer, etc.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

As used herein, the term “specific to” is used to define the relationship between macromolecular binding partners. For example, as used above, two nucleotide sequences that possess total complementarity to one another would be considered “specific” for one another, i.e., each totally complementary nucleotide would be specific to the other.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In the context of the present disclosure, the following abbreviations for the commonly occurring nucleic acid bases are used. “A” refers to adenine, “C” refers to cytosine, “G” refers to guanine, “T” refers to thymine, and “U” refers to uracil. The aforementioned abbreviations may also be used to refer to nucleosides or nucleotides comprising the nucleic acid bases. For example, “G” may refer guanine, guanosine, or guanidine, depending on the context.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1—Protocol

The following non-limiting protocol demonstrates the inventors’ novel method to combine in situ scDNA sequencing with in situ scRNA sequencing.

Some sections of this non-limiting example protocol are adapted from publicly available protocols for Split-Pool Ligation based Transcriptomics sequencing in mammalian and bacterial cells (7, 8). The method of the current disclosure may be performed using several techniques for generating fixed and permeabilized cells and for ligating adapters to DNA sequences known in the art. However, for illustrative purposes the following example protocol is presented.

### Prepare the DNA Barcoding Plates for Later Use

By way of example but not by way of limitation, methods for preparing plates loaded with adapters for ligation are well known in the art. See, e.g., references 7 and 8, incorporated by reference herein in their entirety. Briefly, barcoding plates were generated using the following method. Plates were loaded with barcode and linking oligos. For each ligation plate the barcode and linker oligonucleotides were annealed with the following thermocycling protocol:
1. Heat to 95 C for 2 minutes
2. Ramp down to 20 C for at a rate of −0.1 C/s
3. 4 C

### 1. Fixation and Permeabilization of Cells

Though this example generates fixed and permeabilized yeast cells, one of skill in the art will recognize that fixing and permeabilizing cells of any type (e.g., vertebrate, mammalian, insect, reptile, bird, bacterial, etc.) are known in the art, using e.g., formalin/formaldehyde and enzyme or detergent compositions. Thus, this general method step of generating fixed and permeabilized cells may be substituted for methods suited to the particular cell type of interest without adversely affecting the novel method disclosed herein.

### Fixation

Briefly, by way of example but not by way of limitation, the following method was used to fix the spheroplasts of the previous step. The following solutions were made:
4% formaldehyde solution in 1×PBS and store at 4 C (or on ice).
1×PBS (on ice)
100 mM Tris pH 8.0 (on ice)
10 units/mL zymolyase
0.4% Tween

Cells were pelleted and resuspended in cold formaldehyde. Samples were stored in a 4 C fridge for ˜18 hours. Cells were pelleted and resuspended in 100 mM Tris-HCL. Zymolyase enzyme was mixed with cells and incubated at 37 deg for 15 min. cells were re-suspended in 0.04% Tween. One of skill in the art will understand that, in this exemplary experiment, the chitin digesting enzyme Zymolyase and non-ionic detergent Tween are used to permeabilized cells. However, other detergents and enzymes are known in the art and may be substituted for Zymolyase and Tween.

2. Amplify Genomic DNA and/or Gene of Interest Inside of Cells (Day 2) and RNA

The following novel method of step 2 is presented as an example protocol that the Inventors have successfully reduced to practice to amplify genomic DNA and RNA in situ using the model system of brewer's yeast. 8 μL of the gDNA barcoded primer stock was added to a 96 well plate. The plate was covered with an adhesive plate seal until ready for use.

The following phi29 mix was prepared on ice at volumes sufficient to generate a total of 12 uL per reaction: 2.5 uL of 10×phi29 buffer, 0.2 uL of 20 mg/mL BSA, 2.5 uL 40 mM (per base) dNTPs, 1 uL 400 U/mL phi29 polymerase, 5.8 uL crowding agent (27% PEG8000, 1.8M trehalose, or 2M sorbitol).

12 μL of the phi29 mix was added to each of the 96 wells. Each well thus contained a volume of 20 uL. Cells were vortexed and 5 μL of cells in 1×PBS were added to each of the 96 wells, for a volume of 25 uL per well. Plates were placed into a thermocycler with the following protocol: (a) 30 C for 16 hrs; (b) 4 C indefinitely.

Phi29 was then quenched via heat inactivation by warming the reactions to 65 C for 10 minutes.

8 μL of the cDNA barcoded primers were then added to each of the 96 wells, in addition to 20 U/uL Maxima H Minus reverse transcriptase, 1×RT buffer, 0.25 U/uL Enzymatics and Superase RNAse inhibitors, and additional crowding agent. The plate was placed back in the thermocycler with the following protocol: 23 C for 10 minutes, 50 C for 50 minutes, 4 C indefinitely. The contents of the plate were then combined in a 5 mL centrifuge tube with 0.1% Triton-X100 and spun at 3000G for 5 minutes. The supernatant was removed and the cells were resuspended in 2 mL of 1×PBS plus RNAse inhibitors. Cells were vortexed and passed through a 15 micron filter.

3. Ligation of Barcodes that Track Single Cells

The following method of barcoding cells is adapted from references 7 and 8. However, it will be apparent that other methods to barcode cells are well known in the art. Briefly, by way of example but not by way of limitation, the following ligation master mix was created: 770 uL molecular grade water, 500 uL 10×T4 ligase buffer, 750 uL 50% PEG8000, 20 uL 400 U/uL T4 DNA ligase. Cells were added to the master mix and incubated. The round 2 blocking solution was made as follows: 316.8 uL 100 uM BC_0340 (7, 8), 300 uL 10×T4 ligase buffer, 538.2 uL molecular grade water.

The cells were then split and pooled and ligated to the round 2 barcodes. The round 2 blocking solution is added to the wells, and incubated. The cells were then split, pooled, and ligated to the round 3 barcodes, wherein the barcodes now comprised the affinity moiety biotin. Finally, the round 3 blocking solution was added to the cells comprised of: 369 uL 100 uM BC_0066 (7, 8), 800 uL 0.5M EDTA, and 2031 uL molecular grade water.

Next, ligation was terminated and the cells were washed in wash buffer (4000 uL 1×PBS and 40 uL 10% Triton X-100) and cells were allocated to sublibraries. The cells were then lysed and the DNA was prepared for sequencing. Many methods of lysing cells are known in the art and it will be apparent to one of skill in the art that substitutions to the reagents used in the method of references 7 and 8 may be made.

By way of example, 2× lysis buffer was made as follows (50 uL per sublibrary): 1 uL 1M Tris-HCl pH 8, 4 uL 5M NaCl, 10 uL 0.5M EDTA, 22 uL 10% SDS, 13 uL molecular grade water.

The cells in each sublibrary were lysed in the lysis buffer with 5 uL proteinase K for 2 hours at 55 C.

Purification of Barcoded gDNA and cDNA:

The following example protocol for purification of barcoded gDNA and cDNA was used. However, it will be apparent to one of skill in the art that substitutions may be made to the following protocol that will not negatively affect the ability of the artisan to practice the method. Furthermore, additional methods of DNA purification of barcoded DNA are well known in the art and the following method is presented as a non-limiting example. Briefly, 5 uL 100 uM PMSF was added to the lysate to terminate the proteinase K. Then, MyOne C1 Dynabeads were incubated with the lysate and allowed to bind to the streptavidin. The lysates were then washed with buffers containing Tris-HCL pH 8, NaCl, EDTA, and nuclease free water to remove unbound molecules.

### Template Switch Reaction:

Because the reverse transcription enzyme poly-cystinates the 3′ end of the cDNA, a primer adapter oligo with ribo-G's on the 3′ end can be used during a subsequent reverse transcription reaction to add a terminal PCR primer adapter to the bead-bound cDNA molecules. The beads were resuspended in the following reverse transcription reaction per sublibrary: 99 uL water, 44 uL 5× buffer, 33 uL PEG8000, 22 uL 10 mM dNTPs, 5.5 uL RNAse inhibitor, 5.5 uL template switch oligo, 11 uL Maxima RNAseH Minus reverse transcriptase. The beads were incubated at room temperature with gentle shaking for 30 minutes and then at 42 C with gentle shaking for 90 minutes. They were then placed against a magnetic rack, the supernatant removed, and resuspended in 100 mM Tris-HCL. The beads were stored at 4 C for no more than 2 days.

4. Addition of a Terminal PCR Primer Adapter to Variable Ends of Bead Bound gDNA:

The following novel methods are presented as example protocols that the Inventors have successfully reduced to practice to extend bead bound gDNA using the model system of brewer's yeast.

### Example Embodiment 1, Amplification of a Known Region Downstream of the ROI(s)

If the gDNA is amplified from an ROI and the downstream sequence(s) of the region(s) is known, one can design a reverse primer that will exponentially amplify the barcoded gDNA off the beads in conjunction with the PCR primer to the 5′ end of the barcode using a high fidelity polymerase such as Q5, Kapa HiFi, etc.

### Example Embodiment 2: Blunt End Ligation of a PCR Primer Adapter to the Terminal End

If you are confident your bead bound gDNA molecules are double stranded, such as if you use a non-barcoded reverse primer during the in situ gDNA amplification to generate more template, a terminal PCR primer adapter can be added through blunt end ligation.

### Anneal Adapters

The following mix was added to a thermocycler at −0.1 C/s from 95 C to 20 C:
a. 1 uL 1M NaCl
b. 9.5 uL BC_108a (100 uM)
c. 9.5 uL BC_109 (100 uM)

### Adapter Ligation

The adapter ligation mix was made as follows per reaction: 17.5 uL nuclease free water, 20 uL WGS Enzymatics ligation buffer, 10 uL WGS Enzymatics DNA ligase, 2.5 uL annealed adapters. Mix was added to the beads and incubated for 15 minutes at 20 C.

Samples were placed against a magnetic rack until the liquid becomes clear. Supernatant was removed and the beads were washed with 250 μL of water, and resuspended in 50 μL of water. 50 μL of the adapter mix was added to the 50 uL sample for a 100 uL reaction volume. Samples were incubated at 20° C. for 15 minutes (lid temperature 30° C.). To stop the reaction, samples can again be placed against a magnetic rack until liquid becomes clear. Samples can then be resuspended in 250 uL Tris-Tween and stored at 4 C for no more than 2 days.

### Example Embodiment 3: Additional Phi29 Reaction with Random Hexamer Primers Appended with a Terminal PCR Primer Adapter

If you are working with single-stranded gDNA from the full genome or ROI(s) with unknown downstream sequences, you can perform a phi29 reaction with the bead-bound gDNA using random hexamer primers with a terminal PCR primer adapter. The random hexamers will bind along the length of each bead-bound gDNA. However, since phi29 and other isothermal polymerases are strand displacing, only the primer bound closest to the 3′ end of the gDNA molecule will remain attached, as all primers being amplified downstream will be kicked off by the upstream enzyme. The final reaction is placed against a magnetic rack and the unbound, failed amplicons are washed away. The complementary strands attached to the bead-bound gDNA now contain both forward and reverse PCR primer adapters and traditional PCR can be performed.

The following phi29 mix was prepared per sample: 5 uL 10×phi29 buffer, 0.5 uL 20 mg/mL BSA, 5 uL 40 mM (per base) dNTPs, 2 uL phi29, 2 uL 10 uM BC_0062 (7, 8), 35.5 uL 2M sorbitol.

Samples were placed against a magnetic rack and until liquid cleared. With sample still on magnetic rack, supernatant was removed, and the samples were washed with 250 μL of water. Samples were then resuspended in 50 μL of phi29 mix and incubated for 1 hour at 30 C.

Amplification of Bead-Bound gDNA and cDNA to Create Bead-Free Copies:

The following PCR mix was prepared per sample: 121 uL Kapa HiFi 2× master mix, 9.68 uL 10 uM BC_0108 and BC_0062 (7, 8), 101.64 uL nuclease free water.

Samples were placed against a magnetic rack until liquid became clear. Samples were washed with 250 uL nuclease-free water. Samples were resuspended with 220 uL PCR mix and split equally into 4 different PCR tubes. The following thermocycling program was then run: (a) 95 C 3 min; (b) 98 C 20 s; (c) 65 C 45 s; (d) 2 C 3 min; Repeat (b-d) 19x (20 total cycles); 4 C hold.

The resulting products can be run on an agarose gel or otherwise analyzed. There will likely be a combination of DNA and primer dimer present.

### SPRI Size Selection (0.8×):

PCR reactions were combined into a single tube. 180 uL of the pooled PCR reaction was removed and placed in new 1.7 mL tube. 144 uL of Kapa Pure Beads were added to tube and vortexed briefly to mix. Samples were incubated for 5 min to bind DNA. Tubes were then placed against a magnetic rack until liquid became clear. Supernatant was removed, and beads were washed 2× with 750 uL 85% ethanol. Ethanol was removed and the beads were air dried bead (˜5 min). Dry beads were then resuspended in 20 μL of water. Once beads were fully resuspended in the water, samples were incubated at 37 C for 10 min. Tubes were then placed against a magnetic rack and until liquid cleared. 18.5 uL of elutant was transferred into a new optical grade PCR tube, and a bioanalyzer trace was run on 10 μL of the elutant. If no dimer is present after size selection, move directly to the next section. If dimer is still present, go back to the previous section and perform another size selection.

### 5. Standard NGS Library Preparation:

Following the novel amplification of bead-bound DNA, the free barcoded genomic DNA sequences were used to create libraries for sequencing according to methods known in the art. At this point, the sublibraries exist as pools of DNA fragments that can be prepared following the user's preferred NGS library preparation protocols. Sequencing adapters may be added using the WGS fragmentation and ligation protocol as described in Kuchina and Brettner et al. (8), tagmentation, etc. One of skill in the art will appreciate that many methods for preparation of libraries for sequencing and sequencing thereof may be substituted for the method described in (8).

### NGS Sequencing:

An Illumina paired-end sequencing run with at least 86 bp in Read 2 was used.

### REFERENCES

1. A. B. Rosenberg*, C. M. Roco*, R. A. Muscat, A. Kuchina, P. Sample, Z. Yao, L. Gray, D. J. Peeler, S. Mukherjee, W. Chen, S. H. Pun, D. L. Sellers, B. Tasic, G. Seelig, Single-cell profiling of the developing mouse brain and spinal cord with split-pool barcoding. Science, eaam8999 (2018).
2. A. Kuchina*, L. M. Brettner*, L. Paleologu, C. M. Roco, A. B. Rosenberg, A. Carignano, R. Kibler, M. Hirano, R. W. DePaolo, G. Seelig, Microbial single-cell RNA sequencing by split-pool barcoding. Science. 371 (2021), doi: 10.1126/science.aba5257.
3. A. C. Payne*, Z. D. Chiang*, P. L. Reginato*, S. M. Mangiameli, E. M. Murray, C. Yao, S. Markoulaki, A. S. Earl, A. S. Labade, R. Jaenisch, G. M. Church, E. S. Boyden, J. D. Buenrostro, F. Chen, In situ genome sequencing resolves DNA sequence and structure in intact biological samples. Science. 371 (2021).
5. US20200263234A1 Entitled: “IN SITU COMBINATORIAL LABELLING OF CELLULAR MOLECULES”
6. U.S. Pat. No. 10,900,065B2 Entitled: “METHODS AND KITS FOR LABELLING CELLULAR MOLECULES”
7. patent application Ser. No. 16/949,949 filed Nov. 20, 2020 Entitled: “A METHOD FOR PREPARATION AND HIGH-THROUGHPUT MICROBIAL SINGLE CELL RNA SEQUENCING OF BACTERIA” Inventors: Georg Seelig, Anna Kuchina, Leandra Brettner, & William DePaolo
8. Rosenberg and Roco et al. Science. 13 Apr. 2018: Vol. 360, Issue 6385, pp. 176-182.
9. Kuchina and Brettner et al. Science. 2021 Feb. 19; 371 (6531).
10. Levy, Blundell, Venkataraman, Petrov, Fisher, Sherlock. Quantitative evolutionary dynamics using high resolution lineage tracing. Nature. 2015 Mar. 12; 519 (7542): 181-6.
11. Kiseleva, Allen, Rutherford, Murray, Morozova, Gardiner, Goldberg, Drummond. A protocol for isolation and visualization of yeast nuclei by scanning electron microscopy (SEM). Nature Protocols. August 2007. 2. 1943-1953.
12. Yin Y, Jiang Y, Lam K G, Berletch J B, Disteche C M, Noble W S, Steemers F J, Camerini-Otero R D, Adey A C, Shendure J. High-Throughput Single-Cell Sequencing with Linear Amplification. Mol Cell. 2019 Nov. 21; 76 (4): 676-690.e10. doi: 10.1016/j.molcel.2019.08.002. Epub 2019 Sep. 5. PMID: 31495564; PMCID: PMC6874760.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method comprising:
a) contacting a plurality of fixed and permeabilized cells comprising genomic DNA and cellular RNA with (i) a first set of DNA amplification primers configured to amplify genomic DNA, and (ii) a DNA polymerase; wherein the DNA amplification primers comprise a design selected from (a) or (b), or a combination of (a) and (b) to generate DNA amplicons:
(a)
(i) a first universal linker sequence (1-ULS); wherein each primer comprises the same 1-ULS sequence;
(ii) optionally, a first well-specific barcode sequence (1-BC); wherein the primers in each well comprise a different 1-BC sequence;
(iii) random hexamers which hybridize to complementary sequences on genomic DNA of the cells;
(b)
(i) the first universal linker sequence (1-ULS); wherein each primer comprises the same 1-ULS sequence;
(ii) optionally, the first well-specific barcode sequence (1-BC); wherein the primers in each well comprises a different 1-BC sequence;
(iii) a sequence that is designed to hybridize to a specific sequence on the genomic DNA of the cell;
and amplifying the DNA;
b) quenching the polymerase used for the amplifying of step a);
c) contacting the plurality of fixed and permeabilized cells with a mixture comprising
(i) reverse transcriptase;
(ii) a first set of reverse transcription primers wherein the set comprises
(a) the first universal linker sequence (1-ULS); wherein each primer in the first primer set comprises the same 1-ULS sequence;
(b) a well-specific barcode sequence (2-BC); wherein the primers in each well comprise a different 2-BC sequence, and optionally wherein, the 2-BC sequence is the same as the 1-BC sequence;
(c) a target hybridization region comprising oligo dT sequences or random hexamer sequences;
d) reverse transcribing the RNA present in the cell to generate cDNA;
e) barcoding the products of step a) and d) with sequential rounds of split and pool to uniquely label the amplified nucleic acid of each cell, wherein the last round of barcoding comprises ligating an oligonucleotide comprising a ULS, a well-specific barcode, an affinity moiety, and common primer sequence to generate barcoded amplicons comprising the affinity moiety and barcoded cDNA comprising the affinity moiety;
f) lysing the plurality of cells to release the barcoded amplicons comprising the affinity moiety and the barcoded cDNA comprising the affinity moiety;
g) capturing the released amplicons by contacting the barcoded amplicons comprising the affinity moiety and the barcoded cDNA comprising the affinity moiety with an affinity capture reagent.

2. The method of claim 1, further comprising performing a template switch reaction by contacting the captured amplicons and cDNA with
(i) reverse transcriptase, and
(ii) template switch primers comprising at least three consecutive riboguanosine nucleotides and a terminal primer sequence.

3. The method of claim 1, further comprising generating fully double-stranded captured amplicons and cDNA, wherein the fully double-stranded amplicons and cDNA comprise the terminal primer sequence.

4. The method of claim 3, wherein generating fully double-stranded captured amplicons comprises contacting the captured barcoded amplicons and cDNA with primers that hybridize to the specific sequence on the amplicons, and a DNA polymerase.

5. The method of claim 3, wherein generating fully double-stranded captured amplicons comprises ligating a double-stranded DNA sequence comprising the terminal primer sequence to the free end of the captured amplicons.

6. The method of claim 3, wherein generating fully double-stranded captured amplicons comprises contacting the captured barcoded amplicons and cDNA with an enzyme comprising polymerase activity, and oligonucleotides, wherein the oligonucleotides comprise random hexamers and the terminal primer sequence, wherein the oligonucleotides are configured to produce double-stranded barcoded amplicons comprising the terminal primer sequence.

7. The method of claim 3, further comprising amplifying the fully double-stranded amplicons and cDNA to generate free amplification products.

8. The method of claim 7, further comprising sequencing the free amplification products.

9. The method of claim 6, wherein the set of primers are selected from only design (a).

10. The method of claim 5, wherein the set of primers are selected from only design (b).

11. The method of claim 4, wherein the set of primers are selected from a combination of designs (a) and (b).

12. The method of claim 1, wherein the amplification of step a) comprises isothermal amplification.

13. The method of claim 12, wherein the temperature of the isothermal amplification reaction is about 20-40° C.

14-18. (canceled)

19. The method of claim 1, wherein step a) comprises contacting the plurality of fixed and permeabilized cells with an isothermal polymerase.

20. The method of claim 1, wherein step a) comprises contacting the plurality of fixed and permeabilized cells with phi29 polymerase.

21. (canceled)

22. The method of claim 1, wherein step a) comprises contacting the plurality of fixed and permeabilized cells with a crowding agent.

23-29. (canceled)

30. The method of claim 1, wherein the oligonucleotides of step e) are ligated to the products of a) and d) with T4 DNA ligase.

31-32. (canceled)

33. The method of claim 1, wherein the affinity moiety and capture reagent of step g) comprise biotin and streptavidin.

34. (canceled)

35. The method of claim 3, wherein generating fully double-stranded captured amplicons and cDNA comprises contacting the captured barcoded amplicons and cDNA with phi29 polymerase.

36-52. (canceled)

53. The method of claim 7, wherein the free amplification products are purified using solid phase reversible immobilization (SPRI) selection.


SEQUENCE LISTING (2 sequences)

SEQ ID NO: 1 - DNA, 35 bp, synthetic construct
  source 1..35: mol_type = other DNA
atccacgtgcttgagactcgtaannnnnnataagc

SEQ ID NO: 2 - DNA, 21 bp, synthetic construct
  source 1..21: mol_type = other DNA
ttaatatggactaaaggaggc